(19) United States
(12) Patent Application Publication
(10) Pub. No.: US 2004/0191315 A1
(43) Pub. Date: Sep. 30, 2004

(54) Office products containing antimicrobial agent

(76) Inventors: Mike Slattery, Yonkers, NY (US); Carl Froelich, Smithtown, NY (US); Peter Adams, Pembroke, MA (US); Robin McLaughlin, Avon, MA (US); Cece Regal-Maxson, Fountain Valley, CA (US)

Correspondence Address: WINSTON & STRAWN, PATENT DEPARTMENT, 1400 L STREET, N.W., WASHINGTON, DC, 20005-3502, US

(21) Appl. No.: 10/410,183

(22) Filed: Apr. 10, 2003

Related U.S. Application Data
(60) Provisional application No. 60/457,237, filed on Mar. 24, 2003.

(51) Int. Cl. A61K 31/53; A61K 31/4162; A61K 9/14

(52) U.S. Cl. 424/486; 514/241; 514/389; 523/122

(57) ABSTRACT

An office product which has at least one exposed surface that is subject to infectious microorganisms, and an antimicrobial agent associated with the exposed surface to render ineffective or destroy infectious microorganisms. The antimicrobial agent may be present on the exposed surfaces of the office product as a coating, or as an integral part of a material used to form the exposed surface(s) of the office product. The antimicrobial agent is ineffective until activated using. Preferably, the antimicrobial agent is an amine which can be activated by a halogen. The present invention also relates to a process of making an infectious microorganism resistant office product which comprises providing the office product with an antimicrobial agent.

### BACKGROUND ART

The present invention relates to an office product which includes an antimicrobial agent that can be activated prior to use in order to prevent contamination by carrying or transporting infectious microorganisms around on the product. The present invention further relates to a method of making such office products.

The exposed surfaces of an office product provide an environment where infectious microorganisms can accumulate. People entering or exiting an office might be carrying those microorganisms with them on a particular day. Furthermore, movement of an office product within the office can provide opportunities to be contacted with additional microorganisms. This is especially true in an office where there is significant traffic and where there is the possibility of exposure to such microorganisms, such as the office of a medical doctor or a dentist, or in a hospital, waste collection facility or similar location. The office products are often moved from location to location, as a result those microorganisms are transported with them and arrive at the new location. Although the contamination of office products by infectious microorganisms poses a serious threat to human health, no particular cleaning or sanitary measures are undertaken or even proposed to prevent this from happening.

Currently, antimicrobial agents for producing a sterile environment do not exist or are severely limited in their antimicrobial abilities. Most commonly used water-soluble antimicrobial agents which contain free halogen have severely limited lifetimes, have adverse impacts to their environment, and produce toxic by-products. Antimicrobial agents which do not contain free halogen, such as quaternary ammonium and phenolic compounds, are only effective towards specific organisms and are water soluble, and can cause skin and eye irritation. They also can be removed from the article due to handling or contact with moisture.

Currently only a few antimicrobial agents have been utilized on the exposed surfaces of various articles, and most of these focus on quaternary ammonium compounds (QACs) anchored on polymer backbones which are then cast as films. Although these films are antimicrobial, they need long contact times to kill the organisms, and surfaces containing these compounds cannot be reactivated once the antimicrobial activity is lost. In addition, such films are relatively expensive to make, and are partially water soluble. These features limit the use of such films in office products.

U.S. Pat. No. 5,902,818 discloses cyclic N-halamine biocidal monomers and polymers and methods of using the same as biocides. The polymeric N-halamine biocidal compounds of the U.S. Pat. No. 5,902,818 patent were prepared by reacting the corresponding unhalogenated polymers with a source of chlorine or bromine. Once a surface becomes ineffective at killing microorganisms due to the inactivation of the N—Cl or N—Br moieties, it can be regenerated by wiping it with an aqueous solution of free halogen. The patent further teaches creating or regenerating the cyclic N-halamine biocide in situ by adding a stoichiometric amount of free halogen, either chlorine or bromine, to a precursor cyclic amine contained in a material such as in paint, oil, textile fabric or the like, or bound to a surface of a material such as wood, glass, plastic polymer coating, textile fabric, metal, rubber, concrete, cloth bandage, or the like. Example 21 of the reference discloses casting unchlorinated poly N-halamine compounds into thin films and then chlorinating them to achieve biocidal efficacy. Specifically, a surface is chlorinated with a diluted solution of sodium hypochlorite by soaking the surface in the aqueous solution for 20 minutes. The surface was then removed, washed with synthetic chlorine-demand-free water, and dried at room temperature overnight to ensure all residual sodium hypochloride was removed.

The U.S. Pat. No. 5,902,818 patent is a general disclosure of the use of halamines as biocides, but an immersion procedure is not generally suitable as a practical approach for treatment of office products. One of the disadvantages is the possibility of the product losing its activity before use due to the inactivation of the N—Cl or N—Br moieties. A washing step like the one disclosed in Example 21 of the patent is not applicable to office products, since they cannot be immersed without damaging the paper or metal components of the office product. This would cause a serious drawback in that the antimicrobial activities of the agents could get lost before the products even get to the customers. In addition, washing in a halogenated solution by individual customers is impractical in the case of office products due to variations in shapes and sizes of the office products. Furthermore, washing office products in a halogen containing solution might cause damage to such products.

The application of a biocide to stock materials is generally known. U.S. Pat. No. 5,716,563 discloses the application of a biocide to a composite panel of wood flakes. This is directed to a foaming composition that is applied to smooth the surfaces of a pressed wood flake board so as to render it suitable for use as an underlayer for flooring. The foaming composition can include anti-fungal or anti-bacterial agents.

Other construction materials, such as wood for use in home building, can be treated to protect against attack by microorganisms. Of course, unlike office products, these materials are installed permanently and generally are not transported after installation.

It is also known to treat paper sheets, recording sheets or transfer sheets with a biocide to prolong their lives. One can refer to U.S. Pat. Nos. 6,137,516, 6,277,229 and 5,663,004. Generally, the amount of biocide added is to protect the sheets from attack. Furthermore, the biocide is considered as an optional additive and is often omitted when cost is an issue.

Thus, there is a need for an office product which includes thereon an antimicrobial agent that can be activated prior to usage of the product in order to prevent contamination by infectious microorganisms when the office product is handled by individuals. This need is now satisfied by the present invention.

### SUMMARY OF THE INVENTION

The present invention relates to an office product for holding or retaining office items which includes an antimicrobial agent associated with the exposed surface(s) of the office product. The inclusion of an antimicrobial agent renders ineffective the infectious microorganisms which the surface(s) of the office product is exposed to.

The office products contemplated by the present invention includes a file folder, a box file, a file jacket, a binder, an envelope, a portfolio, or even a hole puncher or a stapler.

The antimicrobial agents can be present on the exposed surface(s) of the office product as a coating, or as an integral part of a material used to form the exposed surface(s) of the office product.

Preferably, the antimicrobial agent that is present in or upon the surface(s) of the office product comprises a compound which is ineffective until activated. More preferably, the antimicrobial agent is an amine which can be activated by a halogen.

The present invention further relates to a process of making an infectious microorganism resistant office product which comprises providing the office product with an antimicrobial agent.

### DETAILED DESCRIPTION OF THE PREFERRED EMBODIMENT

The present invention relates to an office product comprising an article for holding or retaining office items and an antimicrobial agent associated with the exposed surface(s) to render ineffective or destroy infectious microorganisms.

“Antimicrobial activities” describe the killing of, as well as the inhibition of, the growth of a broad spectrum of pathogenic microorganisms such as bacteria, yeast, fungi, algae, viruses, and mold. Moreover, the microbiocidal activities of the office products of the present invention are readily regeneratable.

“An office product” as used in the present invention means a product that can be used in an office setting and which has at least one exposed surface that is subject to infectious microorganisms. The office product may be capable of holding or retaining office items, such as supplies or information in electronic or printed form. Examples of the office product can include a file folder, a box file, a file jacket, a binder, an envelope, a portfolio, a hole puncher, or a stapler, a floppy disc, a compact disc.

The antimicrobial agent can be applied to a surface of the office products which is subject to infectious microorganisms to impart antimicrobial properties to the surface. This can be achieved by any means, for example, by coating, spraying, spreading, dipping, or brushing. Such surfaces include, but are not limited to, surfaces made of cellulose, fiberglass, plastics, metals, glass, ceramic, wood, leather, cloth, and painted surfaces.

Coating the exposed surface(s) with an antimicrobial agent can be achieved by various methods. Examples of useful methods of coating the exposed surface(s) include 1) wiping the exposed surface(s) with a cloth, pad or other substrate that contains an aqueous suspension of the antimicrobial agent, or 2) spraying the exposed surface(s) with a solution or suspension of the antimicrobial agent. The time of exposure of the exposed surface(s) to the antimicrobial agent can vary from a few seconds to hours, days or longer depending on the application. This process may be repeated to apply additional recharging of the antimicrobial agent on the surface of the product.

Once coated onto the surface in question, the antimicrobial agent of the present invention does not readily leach out, eliminating the need for repeated coatings. In addition, the ability of the antimicrobial agent to strongly bind to the coated surfaces indicates that no significant particulate material is given off from such surfaces during handling or use of the product.

Alternatively, the antimicrobial agent can be made an integral part of a material used to form the exposed surface(s) of the article. For example, the antimicrobial agent can be incorporated into plastics to impart antimicrobial properties to the exposes surface(s) of the article which is made of plastic. Further, the antimicrobial agent can be blended with other solid materials to produce materials such as paper, computer keyboard covers, computer keys and board material to impart antimicrobial properties to those materials if they are used in forming the exposed surface of the article.

It is preferred that the antimicrobial agent used in the present invention is in a form of a precursor and is ineffective until it is activated when it is coated on the surface(s) of or impregnated within the material of the office products. These antimicrobial agents are activated immediately before use by reacting it with an activating agent.

In a preferred embodiment, an amine is used as the precursor of an antimicrobial agent. An “amine,” as used herein, refers to a 4- to 7-membered ring, wherein at least 3 members of the ring are carbon, and from 1 to 3 members of the ring are nitrogen heteroatom, and from 0 to 1 member of the ring is oxygen heteroatom, wherein from 0 to 2 carbon members comprise a carbonyl group, and wherein at least 1 to 3 nitrogen atoms are substituted with a hydrogen or hydroxyalkyl group, such as —CH₂OH, or an alkoxyalkyl group, such as —CH₂OCH₃. In addition, the ring members can be further substituted with alkyl groups, such as methyl, ethyl, etc., or hydroxy groups. Halamines are generally disclosed in U.S. Pat. No. 5,490,983 issued to Worley, et al.

Amines suitable for use in accordance with the present invention include, but are not limited to, the following: monomethylol-5,5-dimethylhydantoin (MDMH), 1,3-dimethylol-5,5-dimethylhydantoin (DMDMH); monomethylolated and dimethylolated derivatives of 2,2,5,5-tetramethyl-1,3-imidazolidin-4-one, 6,6-dimethyl-1,3,5-triazine-2,4-dione, 4,4,5,5-tetramethyl-1,3-imidazolidin-2-one, cyanuric acid and 5,5-dimethylhydantoin; and monomethoxylated and dimethoxylated derivatives of monomethylolated and dimethylolated derivatives of 6,6-dimethyl-1,3,5-triazine-2,4-dione, 4,4,5,5-tetramethyl-1,3-imidazolidin-2-one, cyanuric acid, 5,5-dimethylhydantoin and 2,2,5,5-tetramethyl-1,3-imidazolidin-4-one. Examples of the monomethoxylated and dimethoxylated compounds are monomethoxymethyl-5,5-dimethylhydantoin and 1,3-dimethoxymethyl-5,5-dimethylhydantoin, respectively. In a presently preferred embodiment, monomethylol-5,5-dimethylhydantoin and 1,3-dimethylol-5,5-dimethylhydantoin are the amines employed.

If an amine is used as the precursor of an antimicrobial agent, it is activated with a halogen containing aqueous solution immediate before use. Preferably, the aqueous halogen solution is contained in a prepackaged disposable wiper.

Most significantly, the use of a prepackaged disposable wiper containing a halogen solution in activating the precursors of the antimicrobial agents has the significant advantage that it makes it possible to activate the precursors of the antimicrobial agents right before use, thus solves the problem of the agents getting deactivated before use. Also, it offers great convenience in handling as compared to washing the article in a large quantity of a halogen containing solution. Due to the variation in sizes and shapes of different office products as well as health concerns, it is often impractical and undesirable to require individual users to immerge the entire office product in large quantities of halogen solution. Furthermore, activating the antimicrobial agents by washing in a halogen containing solution would cause great waste since it is unnecessary to coat the surface(s) of the office product which will not be exposed to infectious microorganisms.

The prepackaged disposable wiper pad of the invention is premoistened with an aqueous halogen containing solution, and prepackaged in a flexible air tight container, and can be conveniently carried in a purse or pocket. It supplies an effective mechanism to rid office products with infectious microorganism.

The wiping pad of the invention may include a substrate or cloth portion which comprises any of various known fabrics that are suitable for wiping a surface. Such substrate materials should be capable of absorbing and retaining a substantial quantity of the halogen solution. Such substrates may comprise various compositions such as, paper, cloth of natural or synthetic fiber, or a sponge-like synthetic composition, such as polyurethane foam. Preferably the wiper pad substrate, to render it more desirable from an environmental standpoint, may be biodegradable, i.e., comprises a material whose decomposition is accelerated upon exposure to air.

The wiper of the invention may be of any convenient size and thickness suitable for the purpose and may vary, although, for convenience a wiper sheet or pad having a surface area of from about 4 square inches to about 100 square inches, and a thickness of from about 0.4 inch to about 4 inches is preferred.

The halogen containing solution used in the saturation of the wiper pad of the invention can be aqueous solution of free halogens, such as chlorine and bromine. Preferably, an aqueous solution of chlorine is used. Other chlorine containing solution such as a sodium hypochlorite solution may also be used.

In preparing the prepackaged disposable wiper pad of the invention, any of various suitable methods may be used. For example, the wiper pad may be saturated with the halogen containing solution and then encapsulated or otherwise sealed in an air tight liquid impermeable package, preferably a flexible envelope type packet of a kind that is known for sealing in moisture laden inserts and optionally also opaque to light. Alternatively, the wiper pad of the desired dimension may first be enclosed in a packet or package in a dry condition and thereafter a measured amount of the active solution injected into an aperture in an otherwise sealed package and the aperture sealed; the solution thereafter being absorbed into and saturating the pad.

When it is desirable to use the wiper pad of the invention, it is only necessary to tear open the sealed envelope or other easily openable package containing the premoistened wiper pad and fully wiping the surface, which it is desired to be immune to infectious microorganisms, sufficiently to assure that the surface is satisfactorily treated, i.e., coated with the wiper pad halogen containing solution with which the pad is saturated.

In another preferred embodiment, a spray bottle containing the halogen solution instead of a prepackaged disposable wiper is used to bring halogen solution into contact with the antimicrobial agent precursor. The structures of a suitable spray bottle have been taught by numerous patents. One particular suitable bottle structure is disclosed to U.S. Pat. No. 6,223,380 to Klima et al (“Klima”). Klima teaches a spray bottle dispensing apparatus which comprises a wiper applicator in addition to other components typical of a spray bottle. The spray bottle dispensing apparatus of Klima has the advantage that the wiper applicator portion displaces the droplets which make up the spray pattern and spreads each droplet over the surface to which the product has been applied. This promotes higher efficiency of the product applied and thus minimizes wastes.

Preferably, the spray bottle can be sealed between uses in order to reduce loss of halogen by evaporation.

The present invention further provides a method to regenerate the antimicrobial agents. This is significant because the exposed surface(s) of the office products in the present invention may become ineffective at killing microorganisms due to inactivation of N—Cl or N—Br moieties. Regeneration can be achieved by the above mentioned prepackaged disposable wiper or a spray bottle containing halogen containing solution, in addition to their function in activating the precursor of the antimicrobial agents.

What is claimed is:

1. An office product comprising an article for holding or retaining office items and having at least one exposed surface that is subject to infectuous microorganisms, and an antimicrobial agent associated at least with the exposed surface(s) of the article sufficiently to resist removal therefrom and which is present in an amount sufficient to render ineffective or destroy infectuous microorganisms, wherein the agent is associated with the product during manufacture, but which is ineffective until activated.

2. The office product of claim 1 wherein said office items include office supplies or information in electronic or printed form.

3. The office product of claim 1 wherein the article is a file folder, a box file, a file jacket, a binder, an envelope, a portfolio, a hole puncher, or a stapler.

4. The office product of claim 1 wherein the exposed surface(s) of the article comprises paper, pressed board, wood or plastic material, or a combination thereof.

5. The office product of claim 1 wherein the antimicrobial agent is present on the exposed surface(s) of the article as a coating, or as an integral part of a material used to form the exposed surface(s) of the article.

6. The office product of claim 1 wherein the exposed surface(s) of the article comprises plastic and the antimicrobial agent is present within the plastic material.

7. The office product of claim 1 wherein the exposed surface(s) of the article comprises paper or compressed board material, and wherein the antimicrobial agent is impregnated in the material.

8. The office product of claim 1 wherein the antimicrobial agent comprises a compound that is present in or upon the exposed surface(s) of the office product.

9. The office product of claim 8 wherein the compound is a halamine that is activated by a solution of a halogen to provide an active microbial agent in an amount sufficient to render ineffective or destroy infectuous microorganisms that contact the exposed surface(s) of the office product.

10. The office product of claim 9 wherein the halamine is a halogenated product of monomethylol-5,5-dimethylhydantoin (MDMH), 1,3-dimethylol-5,5-dimethylhydantoin (DMDMH), 5,5-dimethylhydantoin (DMH); monoalkylolated and dialkylolated derivatives of 2,2,5,5-tetramethyl-1,3-imidazolidin-4-one, 6,6-dimethyl-1,3,5-triazine-2,4-dione, 4,4,5,5-tetramethyl-1,3-imidazolidin-2-one, cyanuric acid and 5,5-dimethylhydantoin (DMH); and monoalkylated and dialkylated derivatives of monoalkylolated and dialkylolated derivatives of 2,2,5,5-tetramethyl-1,3-imidazolidin-4-one 6,6-dimethyl-1,3,5-triazine-2,4-dione, 4,4,5,5-tetramethyl-1,3-imidazolidin-2-one, cyanuric acid, and 5,5-dimethylhydantoin, an alkylated derivative thereof or mixtures thereof.

11. The office product of claim 8 in combination with an applicator that comprises a solution for activating or recharging the antimicrobial agent by application to the exposed surface(s) of the article.

12. The office product of claim 11 wherein the antimicrobial agent includes a halamine and the solution includes chlorine for activating the halamine.

13. A method for preventing transmission of infectuous microorganisms due to handling of an office product, which comprises:
determining at least one exposed portion of an office product which is used in a contaminable environment where it can be subjected to contamination by infectuous microorganisms; and
providing the exposed portion(s) of the office product with an antimicrobial agent in an amount sufficient to render ineffective or destroy infectious microorganisms that come in contact with such portion(s), wherein the agent is sufficiently resistant to removal from the exposed portion(s) of the office product and the agent is associated with the product during manufacture, but which is ineffective until activated.

14. The method of claim 13 wherein the antimicrobial agent is associated with the exposed portion(s) of the office product prior to exposure to or contamination by the infectuous microorganisms, or prior to use in the contaminable environment.

15. The method of claim 13 wherein the antimicrobial agent is applied to the exposed portion(s) of the office product as a coating, or as an integral part of a material used to form the exposed portion(s) of the office product.

16. The method of claim 13 wherein the office product includes an article for holding or retaining information in printed or electronic form and is in the form of a file folder, a box file, a file jacket, a binder, an envelope or a portfolio.

17. The method of claim 13 wherein the antimicrobial agent comprises a compound that is present in or upon the exposed portion(s) of the office product, and which further comprises activating the compound to form the antimicrobial agent prior to use of the office product.

18. The method of claim 13 wherein the office product is a file folder, a box file, a file jacket, a binder, an envelope, a portfolio, a hole puncher, or a stapler.

19. The method of claim 13 wherein the antimicrobial agent comprises a halamine and the activating is achieved by contacting the exposed surface(s) of the office product with a halogen.

20. The method of claim 19 which further comprises periodically activating the compound after use of the office product to continue to provide the antimicrobial agent.

21. The method of claim 19 which further comprises providing an applicator that comprises a solution for regeneration of the compound by application to the exposed surface(s) of the article to form the antimicrobial agent.
